(19) United States
(12) Patent Application Publication
(10) Pub. No.: US 2006/0189670 A1
(43) Pub. Date: Aug. 24, 2006

(54) Process for the preparation of anastrozole and intermediates thereof

(75) Inventors: Anil Shahaji Khile, New Panvel (IN); Narendra Shriram Joshi, Navi Mumbai (IN); Shekhar Bhaskar Bhirud, Navi Mumbai (IN)

Correspondence Address: M. CARMEN & ASSOCIATES, PLLC, 170 OLD COUNTRY ROAD, SUITE 400, MINEOLA, NY, 11501, US

(73) Assignee: Glenmark Pharmaceuticals Limited, Mumbai (IN)

(21) Appl. No.: 11/359,248

(22) Filed: Feb. 22, 2006

Related U.S. Application Data
(60) Provisional application No. 60/655,795, filed on Feb. 24, 2005.

(30) Foreign Application Priority Data
Feb. 22, 2005 (IN) 195/MUM/2005

(51) Int. Cl. C07D 249/08; A61K 31/4196

(52) U.S. Cl. 514/383; 548/262.2

(57) ABSTRACT

A process for the preparation of anastrozole is provided, the process comprising:
(a) reacting 3,5-bis(1-cyano-1-methylethyl)benzyl halide with a 4-Z-1,2,4-triazole compound of the formula 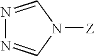 wherein Z is a protecting group to produce 2,2′-[5-(4-Z-1,2,4-triazolium-1-ylmethyl)-1,3-phenylene]di(2-methylpropionitrile) halide; and (b) deprotecting the 2,2′-[5-(4-Z-1,2,4-triazolium-1-ylmethyl)-1,3-phenylene]di(2-methylpropionitrile)halide to produce anastrozole. Also provided is anastrozole substantially free of its isomers.

### PRIORITY

This application claims the benefit under 35 U.S.C. §119 to U.S. Provisional Application No. 60/655,795, filed on Feb. 24, 2005, and entitled “PROCESS FOR THE PREPARATION OF ANASTROZOLE” and Indian Provisional Application No. 195/MUM/2005, filed on Feb. 22, 2005, and entitled “PROCESS FOR THE PREPARATION OF ANASTROZOLE”, the contents of each of which are incorporated by reference herein.

### BACKGROUND OF THE INVENTION

1. Technical Field

The present invention generally relates to an improved process for the preparation of anastrozole and intermediates thereof.

2. Description of the Related Art

Anastrozole, also known as α,α,α′,α′-tetramethyl-5-(1H-1,2,3-triazol-1-ylmethyl) -1,3-benzenediacetonitrile or 2,2′-[5-(1H-1,2,4-triazol-1-ylmethyl)-1,3-phenylene]di(2-methylpropionitrile), is represented by the structure of Formula I.

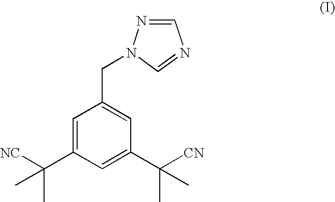

Generally, anastrozole is a potent and selective non-steroidal aromatase inhibitor that lowers serum estradiol concentrations and has no detectable effect on formation of adrenal corticosteroids or aldosterone. Anastrozole is indicated for adjuvant therapy of postmenopausal women having early breast cancer and for the treatment of advanced breast cancer in postmenopausal women with disease progression following tamoxifen therapy. Anastrozole is sold under the brand name Arimidex®. See, e.g., The Merck Index, Thirteenth Edition, 2001, p. 633, monograph 632; and Physician's Desk Reference, “Arimidex,” 58^{th} Edition, pp. 653-658 (2004).

U.S. Pat. No. 4,935,437 (“the ‘437 patent”), herein incorporated by reference, discloses a process for preparing anastrozole. In general, the process disclosed in the ‘437 patent includes reacting 3,5-bis(bromomethyl)toluene with potassium cyanide to produce 3,5-bis(cyanomethyl)toluene, which is then reacted with methyl iodide to provide a 3,5-bis(1-cyano-1-methylethyl)toluene intermediate. Next, this intermediate is reacted with N-bromosuccinimide followed by a reaction with sodium 1,2,4-triazole to produce anastrozole and its isomer, 2,2′-[5-(1,2,4-triazol-4-ylmethyl)-1,3-phenylene]di(2-methylpropionitrile) of Formula II. 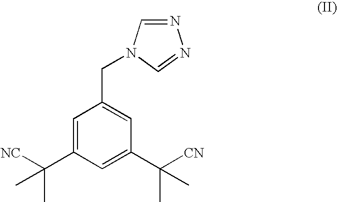
A problem associated with this process is that the isomer of Formula II is an undesired product; thus requiring additional steps to separate it from anastrozole.

Accordingly, there remains a need for an improved process for preparing anastrozole substantially free of undesired isomers.

### SUMMARY OF THE INVENTION

In accordance with one embodiment of the present invention, a compound of Formula III is provided: 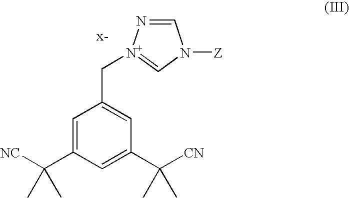
wherein X is a halide and Z is a protecting group.

In accordance with a second embodiment of the present invention, a 2,2′-[5-(4-amino -1,2,4-triazolium- 1-ylmethyl)-1,3-phenylene]di(2-methylpropionitrile)halide of Formula IV is provided: 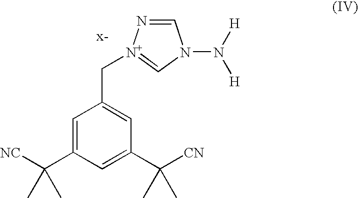
wherein X is a halide.

In accordance with a third embodiment of the present invention, a process for the preparation of a compound of Formula III is provided: 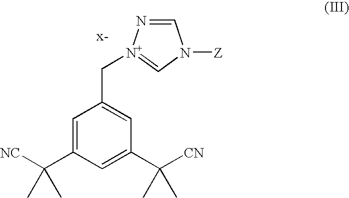
wherein X is a halide and Z is a protecting group, the process comprising reacting a 3,5-bis(1-cyano -1-methylethyl)benzyl halide with a 4-Z-1,2,4-triazole compound of the general formula 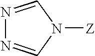
wherein Z has the aforementioned meaning.

In accordance with a fourth embodiment of the present invention, a process for the preparation of anastrozole is provided comprising the steps of:

(a) reacting a 3,5-bis(1-cyano-1-methylethyl)benzyl halide with a 4-Z-1,2,4-triazole compound of the general formula 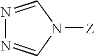
wherein Z is a protecting group to produce a compound of Formula III: 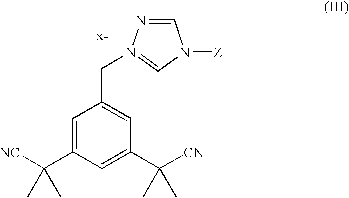
wherein X is a halide and Z has the aforementioned meaning; and

(b) deprotecting the compound of Formula III to produce anastrozole.

In accordance with a fifth embodiment of the present invention, substantially pure anastrozole is provided.

In accordance with a sixth embodiment of the present invention, anastrozole substantially free of its isomers is provided.

In accordance with a seventh embodiment of the present invention, anastrozole substantially free of its 2,2′-[5-(1,2,4-triazol-4-ylmethyl)-1,3-phenylene]di(2-methylpropionitrile) isomer is provided.

In accordance with an eighth embodiment of the present invention, a pharmaceutical composition is provided comprising a therapeutically effective amount of anastrozole substantially free of its isomers.

In accordance with an ninth embodiment of the present invention, a pharmaceutical composition is provided comprising a therapeutically effective amount of anastrozole substantially free of its 2,2′-[5-(1,2,4-triazol-4-ylmethyl)-1,3-phenylene]di(2-methylpropionitrile) isomer.

### DETAILED DESCRIPTION OF THE PREFERRED EMBODIMENTS

One aspect of the present invention provides processes for the preparation of anastrozole and intermediates thereof. In one embodiment, a process of the present invention provides anastrozole substantially free of its isomers. The term “anastrozole substantially free of its isomers” as used herein shall be understood to mean anastrozole formed with little to no isomer content. In this manner, the amount of any isomer of anastrozole, if present, resulting from the process for preparing anastrozole will be in relatively minor amounts, e.g., less than about 0.5 weight percent, preferably less than about 0.05 weight percent and most preferably 0 weight percent of any isomer of anastrozole. In one embodiment, anastrozole is substantially free of its 2,2′-[5-(1,2,4-triazol-4-ylmethyl)-1,3-phenylene]di(2-methylpropionitrile) isomer of Formula II.

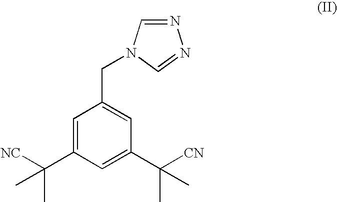

The present invention prepares a novel intermediate which is advantageously useful in the preparation of anastrozole. The novel intermediate is a compound represented by the structure of the Formula III: 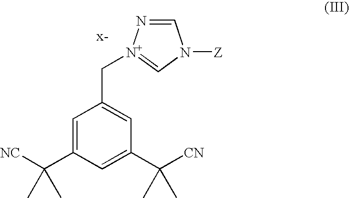
wherein X is a halide, e.g., chloride, bromide and the like, and Z is a protecting group.

In another embodiment, the novel intermediate is a 2,2′-[5-(4-amino-1,2,4-triazolium -1-ylmethyl)-1,3-phenylene]di(2-methylpropionitrile)halide represented by the structure of Formula IV: 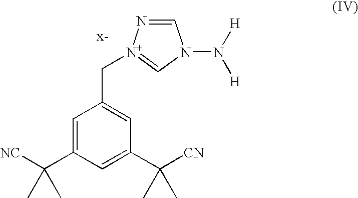
wherein X is a halide.

In general, the novel intermediates can be prepared by reacting a 3,5-bis(1-cyano -1-methylethyl)benzyl halide with a 4-Z-1 ,2,4-triazole of the general formula: 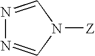
wherein Z is a protecting group. Useful protecting groups include, but are not limited to, amines, e.g., —NH₂, and the like.

The reaction can be carried out by heating the 3,5-bis(1-cyano-1-methylethyl)benzyl halide with a 4-Z-1,2,4-triazole in a solvent. Suitable solvents include, but are not limited to, water, alcohols, e.g., alkyl alcohols, aryl alcohols, arylalkyl alcohols and the like, ketones, e.g., alkyl ketones, arylalkyl ketones and the like, nitrites, N,N-dimethylformamide, dimethylsulfoxide and the like. Representative examples of alcohols include methanol, ethanol, n-propanol, isopropanol, 2-propanol, n-butanol, isobutanol, benzyl alcohol and the like. Representative examples of ketones include acetone, methylisobutyl ketone, methylethyl ketone, acetophenone and the like. Representative examples of nitrites include benzonitrile, acetonitrile and the like. Preferably, the process is preferably performed in an alcohol solvent, and most preferably in 2-propanol.

The temperature of the reaction will ordinarily range from about 20° C. to about 150° C., preferably from about 75° C. to about 100° C., and most preferably from about 80° C. to about 85° C. The time of the reaction may be from about 3 hours to about 12 hours, preferably from about 3 hours to about 10 hours, and most preferably from about 5 hours to about 8 hours. Generally, the molar ratio of 3,5-bis(1-cyano-1-methylethyl)benzyl halide to 4-Z-1,24-triazole can range from about 1:1 to about 1:2 and preferably from about 1:1.4 to about 1:1.6.

The starting materials, intermediates, and compounds of this invention may be isolated and purified using conventional techniques, e.g., filtration, distillation, crystallization, chromatography, and the like. They may be characterized using conventional methods, including physical constants and spectral data. For example, on completion of the reaction of the 3,5-bis(1-cyano-1-methylethyl)benzyl halide and 4-Z-1,2,4-triazole, the reaction mass may be cooled and filtered by conventional techniques. The resulting compound of Formula III, e.g., a 2,2′-[5-(4-amino-1,2,4-triazolium-1-ylmethyl)-1,3-phenylene]di(2-methylpropionitrile)halide, may then be purified by conventional techniques known to those skilled in the art, e.g., crystallization in a suitable solvent, e.g., alcohols, hydrocarbons, ketones and mixtures thereof. Representative examples of alcohols include methanol, ethanol, n-propanol, isopropanol, 2-propanol, n-butanol, isobutanol, benzyl alcohol and the like. Representative examples of hydrocarbons include aliphatic, hydrocarbons, e.g., .n-hexane and the like, aromatic hydrocarbons, e.g., toluene, xylene and the like, and halogenated hydrocarbons, e.g., dichloromethane and the like. Representative examples of ketones include acetone, methylisobutyl ketone, methylethyl ketone, acetophenone and the like. Preferably the solvent is an alcohol, hydrocarbon or mixtures thereof. Preferably, the compound of Formula III is purified by crystallizing in a mixture of 2-propanol and dichloromethane.

The starting 3,5-bis(1-cyano-1-methylethyl)benzyl halide for use in the processes of the present invention is known in the art, e.g., in U.S. Pat. No. 4,935,437, the contents of which are incorporated by reference. In general, the 3,5-bis(1-cyano-1-methylethyl)benzyl halide can be prepared by first reacting mesitylene with an N-halosuccinimide to provide a 3,5-bis(halomethyl)toluene. Suitable N-halosuccinimides include, but are not limited to, N-fluorosuccinimide, N-chlorosuccinimide, N-bromosuccinimde, N-iodosuccinimide and the like. A preferred N-halosuccinimide for use herein is N-bromosuccinimde. The reaction may be carried out by heating the mesitylene and N-halosuccinimide in a solvent. The temperature of the reaction will ordinarily range from about 70° C. to about 150° C., preferably from about 75° C. to about 100° C., and most preferably from about 80° C. to about 85° C. The time of the reaction may be from about 2 hours to about 6 hours, preferably from about 2 hours to about 5 hours, and most preferably from about 2 hours to about 3 hours. Useful solvents include, but are not limited to, haloalkanes, e.g., carbon tetrachloride, dichloromethane, chloroform and the like, alkyl acetates, e.g., methyl acetate, ethyl acetate and the like, and mixtures thereof. A preferred solvent is carbon tetrachloride.

On completion of the reaction, the reaction mass may be separated by techniques well known in the art, such as filtration, preferably at room temperature. If desired, the 3,5-bis(halomethyl)toluene obtained from this reaction may be purified by conventional techniques known to those skilled in the art, e.g., crystallization in a suitable solvent, for example, an alcohol, aliphatic or aromatic hydrocarbon, and/or ketone. Useful alcohols include, but are not limited to, methanol, ethanol, isopropyl alcohol, and the like and mixtures thereof. Useful aliphatic or aromatic hydrocarbons include, but are not limited to, n-hexane, toluene, xylene and the like and mixtures thereof. Useful ketones include, but are not limited to, acetone, methylisobutyl ketone, methyl ethyl ketone and the like and mixtures thereof. Preferably, crystallization is carried out in an alcohol and/or hydrocarbon solvent, and most preferably in a lower alcohol, e.g., methanol.

Next, 3,5-bis(halomethyl)toluene can be reacted with a cyanide-containing radical to provide 3,5-bis(cyanomethyl)toluene. Suitable cyanide-containing radicals include, but are not limited to, potassium cyanide, sodium cyanide, trimethylsilyl cyanide, and the like with sodium cyanide being preferred. The reaction may be carried out by heating the 3,5-bis(halomethyl)toluene and cyanide in a suitable solvent. The temperature of the reaction will ordinarily range from about 35° C. to about 80° C., preferably from about 35° C. to about 50° C., and most preferably from about 40° C. to about 45° C. The time of the reaction may be from about 3 hours to about 14 hours, and preferably from about 3 hours to about 10 hours. Useful solvents include, but are not limited to, water, haloalkanes, e.g., carbon tetrachloride, dichloromethane, chloroform and the like, alcohols, e.g., methanol, ethanol, 2-propanol and the like, and mixtures thereof, e.g., a haloalkane and water or alcohol and water. Preferably the solvent used in this step of the reaction is a mixture of a haloalkane and water, and most preferably a mixture of dichloromethane and water.

On completion of the reaction, the reaction mass may be cooled, extracted and concentrated, e.g., by evaporation. The 3,5-bis(cyanomethyl)toluene obtained may be purified by conventional techniques known to those skilled in the art and discussed hereinabove.

Next, the resulting 3,5-bis(cyanomethyl)toluene is reacted with a methyl halide to provide 3,5-bis(1-cyano-1-methylethyl)toluene. Suitable methyl halides include, but are not limited to, methyl fluoride, methyl chloride, methyl bromide, methyl iodide and the like, with methyl iodide being preferred. The reaction may be performed by heating the 3,5-bis(cyanomethyl)toluene and methyl halide in a solvent. The temperature of the reaction may be in the range of from about 0° C. to about 60° C., preferably from about 0° C. to about 40° C., and most preferably from about 0° C. to about 25° C. The time of the reaction may be from about 3 hours to about 10 hours, preferably from about 3 hours to about 7 hours, and most preferably from about 3 hours to about 5 hours. Useful solvents include, but are not limited to, dialkyl amides, e.g., N,N-dimethylacetamide, N,N-dimethylformamide and the like, aromatic hydrocarbons, e.g., benzene, toluene, xylene, mesitylene and the like, ethers, e.g., diethyl ether, diisopropyl ether, tetrahydrofuran and the like and mixtures thereof. Preferably, the solvent used in this step of the reaction is a dialkyl amide, and most preferably N,N-dimethylformamide.

On completion of the reaction, the reaction mass may be diluted, extracted and concentrated, e.g., by evaporation. The 3,5-bis(1-cyano-1-methylethyl)toluene obtained may be purified by conventional techniques known to those skilled in the art, e.g., crystallization in, for example, alcohols, hydrocarbons and haloalkanes, preferably in alcohols and haloalkanes, and most preferably in carbon tetrachloride.

The 3,5-bis(1-cyano-1-methylethyl)toluene is then reacted with an N-halosuccinimide to provide the 3,5-bis(1-cyano-1-methylethyl)benzyl halide. Useful N-halosuccinimides include, but are not limited to, N-chlorosuccinimide, N-bromosuccinimde, N-iodosuccinimide and the like, with N-bromosuccinimde being preferred. The reaction may be performed by heating the 3,5-bis(1-cyano-1-methylethyl)toluene and N-halosuccinimide in a solvent. The temperature of the reaction will ordinarily range from about 70° C. to about 150° C., preferably from about 75° C. to about 100° C., and most preferably from about 80° C. to about 85° C. The time of the reaction may be from about 2 hours to about 6 hours, preferably from about 2 hours to about 5 hours, and most preferably from about 2 hours to about 3 hours. Useful solvents include, but are not limited to, haloalkanes, e.g., carbon tetrachloride, dichloromethane, chloroform and the like, alkyl acetates, e.g., methyl acetate, ethyl acetate and the like, and mixtures thereof with carbon tetrachloride being preferred.

On completion of the reaction, the reaction mass may be diluted, extracted and concentrated, e.g., by evaporation. The 3,5-bis(1-cyano-1-methylethyl)benzyl halide obtained may be purified by standard techniques known to those skilled in the art, e.g., crystallization in an alcohol, hydrocarbon and/or ketone solvent as discussed hereinabove, preferably in an alcohol and/or hydrocarbon, and most preferably in 2-propanol.

The novel intermediates of Formulae III and IV may then be used to prepare anastrozole substantially free of its isomers. In one embodiment, a process of the present invention prepares anastrozole by deprotecting the intermediate of Formulae III and IV to provide the product anastrozole substantially free of its isomers. As one skilled in the art would readily appreciate, deprotection of the protecting group Z can be carried out by any suitable deprotecting agent depending on the protecting group being used. For example, in the case where the protecting group Z is an amine, for example, NH₂, deprotection can be carried out by deaminating the amine group with a suitable deaminating agent. Suitable deaminating agents include, but are not limited to, cyanides, for example, inorganic nitrites, organic nitrites, nitrous acid, and the like and mixtures thereof. Useful inorganic nitrites may be sodium nitrite, potassium nitrite and the like. Useful organic nitrites may be a C₁-C₆ alkyl nitrite such as methyl nitrite and the like. The preferred deaminating agent is nitrous acid, which may be prepared in situ. For example, in a preferred embodiment, an inorganic nitrite is reacted with a mineral acid to produce the nitrous acid in situ. The most preferred deaminating agent is sodium nitrite and a mineral acid, for example, hydrochloric acid in an aqueous medium.

Deprotection of the 2,2′-[5-(4-Z-1,2,4-triazolium-1-ylmethyl)-1,3-phenylene]di(2-methylpropionitrile) halide, i.e., removing the protecting group, can be carried out at a temperature of from about 0° C. to about 25° C., preferably from about 0° C. to about 15° C., and most preferably from about 0° C. to about 5° C. The time period for removing the protecting group can range from about 3 hours to about 12 hours, preferably from about 3 hours to about 10 hours, and most preferably from about 6 hours to about 9 hours. Generally, a molar excess of the deprotecting agent can be used to deprotect the 2,2′-[5-(4-Z-1,2,4-triazolium -1-ylmethyl)-1,3-phenylene]di(2-methylpropionitrile) halide. In one embodiment, the molar ratio of the deprotecting agent to the 2,2′-[5-(4-Z-1,2,4-triazolium-1-ylmethyl)-1,3-phenylene]di(2-methylpropionitrile) halide can range from about 1:1 to about 4:1 and preferably from about 1.9:1 to about 2:1.

Following the removal of the protecting group, the pH of the reaction mixture may be adjusted to greater than about 8 using a suitable base such as an aqueous ammonia solution. Followed adjusting the pH of the reaction mixture, the reaction mixture may be extracted with an organic solvent, e.g., a halogenated hydrocarbon such as dichloromethane, and the organic layer can then be concentrated by, for example, evaporation, to obtain anastozole. The anastrozole obtained may be further purified by conventional techniques known to those skilled in the art, e.g., crystallization in an alcohol solvent, hydrocarbon solvent, ketone solvent, ester solvent, ether solvent and mixtures thereof. Preferably the solvent is an ester and/or hydrocarbon, and most preferably is a mixture of ethyl acetate and cyclohexane.

In a preferred embodiment of the present invention, anastrozole of Formula I is prepared as generally shown below in Scheme 1.

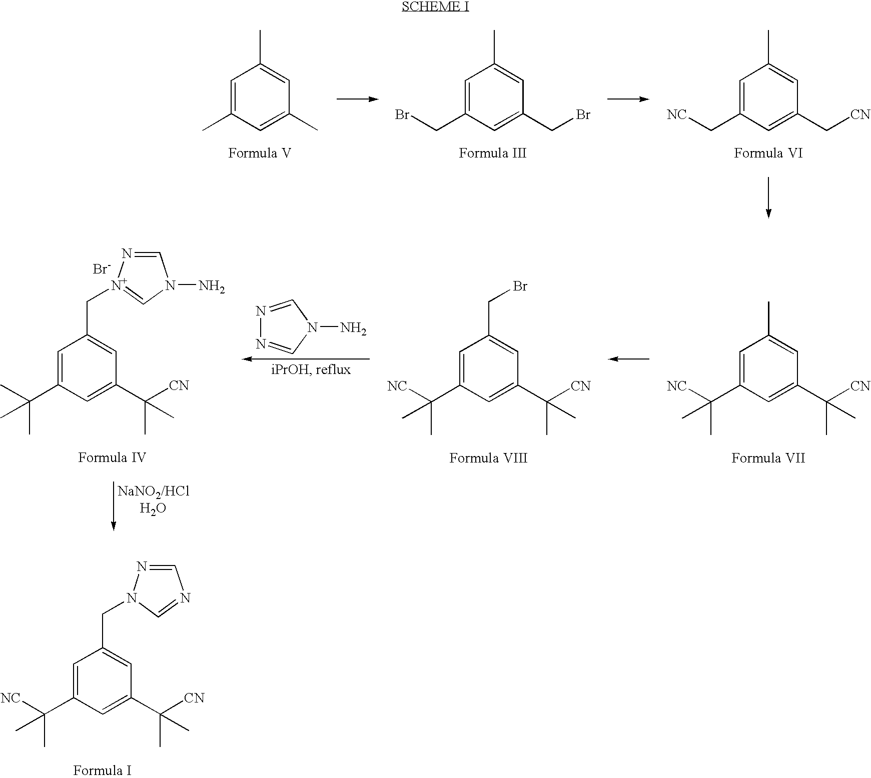

The process of the present invention also prepares substantially pure anastrozole, e.g., a purity greater than about 99%, and preferably greater than about 99.5%. The anastrozole is substantially pure of all isomers including the isomer, 2,2′-[5-(1,2,4-triazol -4-ylmethyl)-1,3-phenylene]di(2-methylpropionitrile) of Formula II. 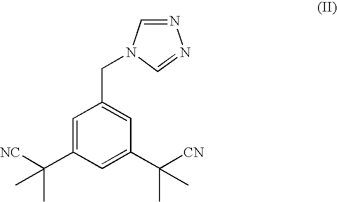
The process may prepare anastrozole that is substantially pure, e.g., having less than about one percent of the undesirable isomer of Formula II.

Anastrozole obtained herein can be of a defined particle size which may be produced by known methods of particle size reduction starting with crystals, powder aggregates and course powder of either crystalline or amorphous anastrozole. For example, a pharmaceutical composition can contain at least micronized anastrozole or a pharmaceutically acceptable salt thereof as obtained herein wherein the D₅₀ and D₉₀ particle size of the unformulated anastrozole or pharmaceutically acceptable salt thereof used is less than about 400 microns, preferably less than about 200 microns, more preferably less than about 150 microns, still more preferably less than about 50 microns and most preferably less than about 15 microns.

Any milling, grinding micronizing or other particle size reduction method known in the art can be used to bring the solid state anastrozole or pharmaceutically acceptable salt thereof into any desired particle size range set forth above. The principal operations of conventional size reduction are milling of a feedstock material and sorting of the milled material by size. For example, a fluid energy mill, or micronizer, is an examplary type of mill known for its ability to produce particles of small size in a narrow size distribution. As those skilled in the art are aware, micronizers use the kinetic energy of collision between particles suspended in a rapidly moving fluid (typically air) stream to cleave the particles. An air jet mill is a preferred fluid energy mill. The suspended particles are injected under pressure into a recirculating particle stream. Smaller particles are carried aloft inside the mill and swept into a vent connected to a particle size classifier such as a cyclone.

Another aspect of the present invention is directed to pharmaceutical compositions containing at least anastrozole or its pharmaceutically acceptable salts obtained herein. The pharmaceutical compositions may be administered orally, parenterally, rectally, transdermally, bucally, or nasally. Suitable forms for oral administration include tablets, compressed or coated pills, dragees, sachets, hard or gelatin capsules, sub-lingual tablets, syrups and suspensions. Suitable forms of parenteral administration include an aqueous or non-aqueous solution or emulsion, while for rectal administration suitable forms for administration include suppositories with hydrophilic or hydrophobic vehicle. For topical administration the invention provides suitable transdermal delivery systems known in the art, and for nasal delivery there are provided suitable aerosol delivery systems known in the art.

Pharmaceutical compositions of the present invention contain anastrozole or its pharmaceutically acceptable salts substantially free of its isomer. In addition to the active ingredient(s), the pharmaceutical compositions of the present invention may contain one or more pharmaceutically acceptable excipients. Suitable excipients and the amounts to use may be readily determined by the formulation scientist based upon experience and consideration of standard procedures and reference works in the field.

Solid compositions of the present invention include powders, granulates, aggregates and compacted compositions. The dosages include dosages suitable for oral, buccal, rectal, parenteral (including subcutaneous, intramuscular, and intravenous), inhalant and ophthalmic administration. Although the most suitable route in any given case will depend on the nature and severity of the condition being treated, the most preferred route of the present invention is oral. The dosages may be conveniently presented in unit dosage form and prepared by any of the methods well-known in the pharmaceutical arts.

The following examples are provided to enable one skilled in the art to practice the invention and are merely illustrative of the invention. The examples should not be read as limiting the scope of the invention as defined in the claims.

### EXAMPLE

Step I—Preparation of 3,5-bis(bromomethyl)toluene

A solution of mesitylene (500 g, 4.16 mol), benzoyl peroxide (53.7 g, 0.22 mol) in carbon tetrachloride (3.75 L) was heated to boiling temperature. To this solution was added N-bromosuccinimide (1480 g, 8.315 mol) in portions over a period of 1 to 1.5 hours at boiling temperature. The reaction mass was further agitated at boiling temperature for 1 hour. The reaction was monitored by thin layer chromatography (“TLC”) (n-Hexane:EtOAc-9.2:0.8). After completion of reaction as determined by TLC, the reaction mass was cooled to room temperature and filtered. The filtrate was washed with sodium sulfite solution (2×500 ml, 5%), sodium carbonate solution (2×500 ml, 5%) and finally with brine (2×1000 ml). The organic layer was concentrated to dryness. The residue obtained was dissolved in methanol (2400 ml) at a temperature of 60° C. to 65° C. and gradually cooled to 10° C. to 15° C. The reaction mass was stirred for 2 hours. The solution was further cooled to a temperature of −5° C. 5° C. to 0° C. and stirred for 2 hours. Next, the stirred solution was filtered, washed with chilled methanol (500 ml), and dried at 35° C. to 40° C. under vacuum to obtain 3,5-bis(bromomethyl) toluene (500 g, 43.23% yield).

Step II—Preparation of 3,5-bis(cyanomethyl)toluene

A solution of 3,5-bis(bromomethyl)toluene obtained in Step I (400 g, 1.44 mol), n-tetrabutyl ammonium bromide (13.3 g, 0.0412 mol), sodium cyanide (176.3 g, 3.597 mol) in a dichloromethane (800.0 ml) and water (400 ml) mixture was refluxed for 8 to 9 hours. The reaction was monitored by TLC (n-Hexane:EtOAc-7.5 :2.5). After completion of reaction as determined by TLC, the reaction mass was cooled to room temperature and the layers were separated. The aqueous layer was extracted with dichloromethane (2×400 ml). The organic layers were combined and washed with water (2×500 ml) and brine (2×500 ml). The organic layer was concentrated to dryness at 40° C. to 45° C. The residue obtained was dissolved in carbon tetrachloride (1000 ml) at 70° C. to 75° C. and gradually cooled to 20° C. to 25° C. The reaction mass was stirred for 2 hours. The solution was further cooled 5° C. to 10° C. and stirred for 2 hours. Next, the stirred solution was filtered, washed with chilled carbon tetrachloride (300 ml), and dried at 40° C. to 45° C. under vacuum to obtain 3,5-bis(cyanomethyl)toluene (229 g, 93.5% yield).

Step III—Preparation of 3,5-bis(1-cyano-1-methylethyl) toluene

A mixture of 3,5-bis(cyanomethyl)toluene obtained in Step II (800 g, 4.70 mol), methyl iodide (2935.2 g, 20.68 mol) and dimethylformamide (11.20 L) was cooled to 0° C. to 5° C. Sodium hydride (60%) dispersion in oil (864.2 g, 36.0 mol) was added in portions over 1 to 1.5 hours. The mixture was then allowed to warm to room temperature and stirred for 2 to 2.5 hours. The reaction was monitored by TLC (n-Hexane:EtOAc-7.5 :2.5). After completion of reaction as determined by TLC, excess sodium hydride was decomposed by adding ethyl acetate. The reaction mass was diluted with water and extracted with dichloromethane (3×5 L). The organic layers were combined and washed with brine (5 L). The organic layer was charcoalized at room temperature for 1 hour and concentrated to dryness at 40° C. to 45° C. The residue obtained was dissolved in carbon tetrachloride (2400 ml) at 70° C. to 75° C. and gradually cooled to 10° C. to 15° C. The reaction mass was stirred for 1 hour. The solution was further cooled to −5° C. to 0° C. and stirred for 3 hours. Next, the stirred solution was filtered, washed with chilled carbon tetrachloride (500 ml), and dried at 40° C. to 45° C. under vacuum to obtain 3,5-bis(1-cyano-1-methylethyl)toluene (748.0 g, 70.3% yield).

Step IV—Preparation of 3,5-bis(l-cyano-1-methylethyl)benzyl bromide

A mixture of 3,5-bis(1-cyano-1-methylethyl)toluene obtained in Step III (600 g, 2.65 mol), N-bromosuccinimide (519 g, 2.916 mol), benzoyl peroxide (17 g, 0.053 mol) and carbon tetrachloride (4.5 L) was refluxed for 2 to 2.5 hours. The reaction mass was cooled and filtered. The filtrate was concentrated to dryness at 40° C. to 45° C. The solid obtained was dissolved in 2-propanol (2 L) at 75° C. to 80° C. and gradually cooled to 10° C. to 15° C. The reaction mass was stirred for 1 hour. The reaction mass was further cooled −5° C. to 0° C., stirred for 2 hours, filtered, and washed with chilled 2-propanol (200 ml). The cake was washed with n-hexane and sucked dry. The cake was then dried at 40° C. to 45° C. under vacuum to obtained 3,5-bis(1-cyano-1-methylethyl)benzyl bromide (700.2 g, 87.0% yield).

Step V—Preparation of 2,2′-[5-(4-amino-1,2,4-triazolium-1-ylmethyl)-1,3-phenylene]di(2-methylpropionitrile)bromide

3,5-bis(1-cyano-1-methylethyl) benzyl bromide obtained in Step IV (600 g, 1.965 mol) was heated with 4-amino-1,2,4-triazole (247.9 g, 2.948 mol) in isopropanol (4.5 L) for 7 to 7.5 hours at 80° C. to 85° C. The reaction mass was gradually cooled to 20° C. to 25° C. The reaction mass was stirred for 1 hour. The reaction mass was further cooled to 0° C. to 5° C., stirred for 2 hours, filtered, and washed with dichloromethane (2×500 ml). The reaction mass was sucked dry. The dried cake was further dried at 40° C. to 45° C. under vacuum to obtained 2,2′-[5-(4-amino-1,2,4-triazolium-1-ylmethyl)-1,3-phenylene]di(2-methylpropio-nitrile)bromide (497.5 g, 65.0% yield).

MP-198-203° C.

IR (In cm⁻¹, KBr): 3204, 3116, 3080, 2987, 2229, 1643, 1607, 1565, 1469, 1438, 1196, 1004 H¹NMR (in CDCl₃,δ (ppm)]: 10.31 (1H,s),9.20 (1H,s),7.66-7.63 (3H,s),6.984 (2H,s),5.657 (2H,s),1.71 ( 12H,s).

M+[309] (free base)

Step VI—Preparation of Anastrozole

A mixture of 2,2′-[5-(4-amino-1,2,4-triazolium-1-ylmethyl)-1,3-phenylene]di(2-methylpropionitrile)bromide obtained in Step V (490 g, 1.258 mol) was suspended in water (4.90 L) and cooled to 0° C. to 5° C. To this suspension, hydrochloric acid (441.3 ml, 5 mol) was added. Next, a solution of sodium nitrite (173.7 gm, 2.517 mol) in water (520 ml) was added at 0° C. to 5° C. for 6 hours. The reaction mass was warmed to room temperature. The reaction mass was maintained at room temperature for 3 to 3.5 hours. Urea (160 g, 2.767 moles) was charged to the reaction vessel at room temperature and stirred for 15 minutes. The reaction mass was cooled to 10° C. to 15° C. and the pH was adjusted to greater than 8 by adding aqueous ammonia solution (400-450 ml). The reaction mass was extracted with dichloromethane (3×1000 ml). The organic layers were combined and washed with water (3×1000 ml) until they were neutral. The organic layer was charcoalized at room temperature for 1 hour and concentrated to dryness at 40° C. to 45° C.. The residue obtained was dissolved in ethyl acetate (490.0 ml) at 60° C. to 65° C. To this solution, diisopropyl ether (2.45 L) was added slowly under stirring. The reaction mass was stirred for 1 hour at 20° C. to 25° C. The reaction mass was further cooled to 10° C. to 15° C. and stirred for 2 hours. The reaction mass was filtered and washed with diisopropyl ether (2×400 ml). The reaction mass was sucked dry. The dried cake was dried at 40° C. to 45° C. under vacuum to obtained anastrozole (300 g, 81.24% yield).

HPLC purity>99.5%

IR(cm⁻¹, KBr): 3435, 3102, 2975, 3048, 2985, 2975, 2236, 1606, 1502, 1476, 1273, 1206, 1138, 1013

H¹NMR (in CDCl₃,δ(ppm)]: 8.17 (1H,s),8.02 (1H,s),7.55 (1H,s),7.34 (2H,d),5.4 (2H,s),1.73 (12H,s).

Mass: M+[294.1] The isomer content of anastrozole as determined by High Performance Liquid Chromatography (HPLC) was less than 0.5%.

While the above description contains many specifics, these specifics should not be construed as limitations of the invention, but merely as exemplifications of preferred embodiments thereof. Those skilled in the art will envision many other embodiments within the scope and spirit of the invention as defined by the Claims and advantages appended hereto.

What is claimed is:

1. A process for the preparation of a compound of Formula III: 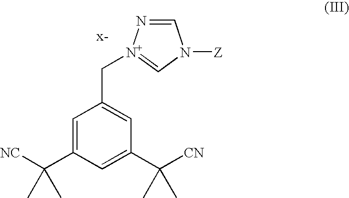
wherein X is a halide and Z is a protecting group, the process comprising reacting a 3,5-bis(1-cyano -1-methylethyl)benzyl halide with a 4-Z-1 ,2,4-triazole compound of the formula 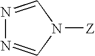
wherein Z has the aforementioned meaning.

2. The process of claim 1, wherein Z is an amine.

3. The process of claim 1, wherein Z is —NH₂.

4. The process of claim 1, wherein the reaction is carried out in a solvent selected from the group consisting of an alcohol, ketone, nitrile, water and mixtures thereof.

5. The process of claim 1, wherein the compound of Formula III is thereafter converted to anastrozole or a pharmaceutically acceptable salt thereof.

6. A compound of Formula III: 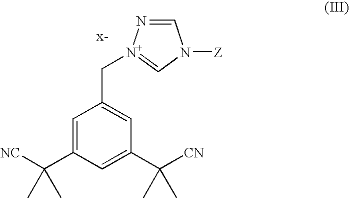
wherein X is a halide and Z is a protecting group.

7. The compound of claim 6, which is a 2,2′-[5-(4-amino-1,2,4-triazolium-1-ylmethyl) -1,3-phenylene]di(2-methylpropionitrile)halide of the general formula: 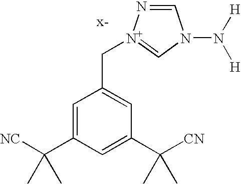
wherein X has the aforestated meaning.

8. The compound of claim 7, wherein X is bromide.

9. A process for the preparation of anastrozole comprising:
(a) reacting a 3,5-bis(1-cyano-1-methylethyl)benzyl halide with a 4-Z-1,2,4-triazole compound of the formula 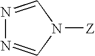
wherein Z is a protecting group to produce a compound of Formula III: 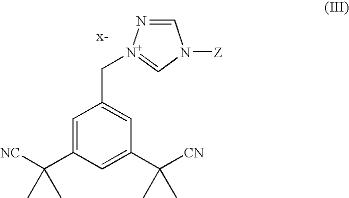
wherein X is a halide and Z has the aforementioned meaning; and
(b) deprotecting the compound of Formula III to produce anastrozole.

10. The process of claim 9, wherein Z is an amine and wherein step (b) comprises deaminating the compound of Formula III.

11. The process of claim 10, wherein the deaminating step comprises removing the protecting group with a deaminating agent.

12. The process of claim 11, wherein the deaminating agent is selected from the group consisting of an inorganic nitrite, organic nitrite, nitrous acid and mixtures thereof.

13. The process of claim 12, wherein the inorganic nitrite is selected from the group consisting of sodium nitrite, potassium nitrite and mixtures thereof.

14. The process of claim 12, wherein the organic nitrite is a C₁-C₆ alkyl nitrite.

15. The process of claim 9, wherein Z is —NH₂ and the step of deprotecting comprises deaminating the compound of Formula III.

16. The process of claim 15, wherein the deaminating step comprises removing the protecting group with a deaminating agent.

17. The process of claim 16, wherein the deaminating agent is selected from the group consisting of an inorganic nitrite, organic nitrite, nitrous acid and mixtures thereof.

18. The process of claim 15, wherein the deaminating step comprises adding an inorganic nitrite with a mineral acid to produce nitrous acid in situ.

19. The process of claim 18, wherein the inorganic nitrite is sodium nitrite and the mineral acid is hydrochloric acid in an aqueous medium.

20. The process of claim 9, further comprising the step of recovering the anastrozole.

21. The process of claim 20, wherein the recovering step comprises basifying with an inorganic base.

22. The process of claim 21, wherein the inorganic base is an aqueous ammonia solution.

23. The process of claim 22, wherein the basification is with an inorganic base to a pH greater than about 8.

24. The process of claim 9, wherein the 3,5-bis(1-cyano-1-methylethyl)benzyl halide of step (a) is prepared by
reacting mesitylene with a N-halosuccinimide to produce a 3,5-bis(halomethyl)toluene;
reacting the 3,5-bis(halomethyl)toluene with a cyanide-containing radical to produce 3,5-bis(cyanomethyl)toluene;
reacting the 3,5-bis(cyanomethyl)toluene with a methyl halide to produce 3,5-bis(1-cyano -1-methylethyl)toluene; and
reacting the 3,5-bis(1-cyano-1-methylethyl)toluene with a N-halosuccinimide to produce 3,5-bis(1-cyano-1-methylethyl)benzyl halide.

25. The process of claim 9, wherein the product anastrozole is substantially free of its isomers.

26. The process of claim 9, further comprising purifying the product anastrozole.

27. Substantially pure anastrozole.

28. The substantially pure anastrozole of claim 27, wherein the anastrozole is substantially free of its isomers.

29. The anastrozole of claim 27, which is substantially free of its 2,2′-[5-(1,2,4-triazol -4-ylmethyl)-1,3-phenylene]di(2-methylpropionitrile) isomer of Formula II.

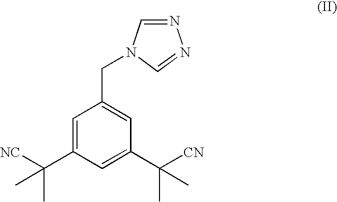

30. The anastrozole of claim 28, having less than one weight percent of isomer impurity.

31. Anastrozole prepared by the process of claim 9.

32. A pharmaceutical composition comprising a therapeutically effective amount of the anastrozole of claim 28 or a pharmaceutically acceptable salt thereof.

33. The pharmaceutical composition of claim 32, wherein the anastrozole is micronized anastrozole or a pharmaceutically acceptable salt thereof having a particle size of less than about 400 microns.

34. The pharmaceutical composition of claim 32, wherein the anastrozole is micronized anastrozole or a pharmaceutically acceptable salt thereof having a particle size of less than about 15 microns.
